(19) United States
(12) Patent Application Publication
(10) Pub. No.: US 2009/0185985 A1
(43) Pub. Date: Jul. 23, 2009

(54) LARGE-PARTICLE CYCLODEXTRIN INCLUSION COMPLEXES AND METHODS OF PREPARING SAME

(75) Inventor: Kenneth J. Strassburger, Cincinnati, OH (US)

Correspondence Address: MICHAEL BEST & FRIEDRICH LLP, 100 E WISCONSIN AVENUE, Suite 3300, MILWAUKEE, WI, 53202, US

(73) Assignee: CARGILL, INCORPORATED, Wayzata, MN (US)

(21) Appl. No.: 12/302,551

(22) PCT Filed: Dec. 5, 2006
(86) PCT No.: PCT/US06/46480

§ 371 Date: Nov. 26, 2008

Related U.S. Application Data
(60) Provisional application No. 60/813,019, filed on Jun. 13, 2006.

(51) Int. Cl. C08B 37/02; A61K 8/49; A23L 1/236

(52) U.S. Cl. 424/49; 536/112; 426/536; 426/535

(57) ABSTRACT

The present invention provides a cyclodextrin inclusion complex comprising a guest encapsulated by cyclodextrin, the complex being greater than about 400 microns in size and methods of making the same. The present invention also provides a method of imparting flavor to a product to form a flavored product, the method comprising: incorporating a large particle cyclodextrin inclusion complex into a product to form a flavored product, the complex comprising a guest encapsulated by a cyclodextrin. The present invention further provides a flavored product comprising a large particle cyclodextrin inclusion complex.

### CROSS-REFERENCE TO RELATED APPLICATIONS

This application claims the benefit of U.S. Provisional Application No. 60/813,019, filed Jun. 13, 2006, which is incorporated by reference herein.

### BACKGROUND

The following U.S. patents disclose the use of cyclodextrins to complex various guest molecules, and are hereby fully incorporated herein by reference: U.S. Pat. Nos. 4,296,137, 4,296,138 and 4,348,416 to Borden (flavoring material for use in chewing gum, dentifrices, cosmetics, etc.); 4,265,779 to Gandolfo et al. (suds suppressors in detergent compositions); 3,816,393 and 4,054,736 to Hyashi et al. (prostaglandins for use as a pharmaceutical); 3,846,551 to Mifune et al. (insecticidal and acaricidal compositions); 4,024,223 to Noda et al. (menthol, methyl salicylate, and the like); 4,073,931 to Akito et al. (nitro-glycerine); 4,228,160 to Szjetli et al. (indomethacin); 4,247,535 to Bernstein et al. (complement inhibitors); 4,268,501 to Kawamura et al. (anti-asthmatic actives); 4,365,061 to Szjetli et al. (strong inorganic acid complexes); 4,371,673 to Pitha (retinoids); 4,380,626 to Szjetli et al. (hormonal plant growth regulator); 4,438,106 to Wagu et al. (long chain fatty acids useful to reduce cholesterol); 4,474,822 to Sato et al. (tea essence complexes); 4,529,608 to Szjetli et al. (honey aroma), 4,547,365 to Kuno et al. (hair waving active-complexes); 4,596,795 to Pitha (sex hormones); 4,616,008 Hirai et al. (antibacterial complexes); 4,636,343 to Shibanai (insecticide complexes), 4,663,316 to Ninger et al. (antibiotics); 4,675,395 to Fukazawa et al. (hinokitiol); 4,732,759 and 4,728,510 to Shibanai et al. (bath additives); 4,751,095 to Karl et al. (aspartamane); 4,560,571 to Sato et al. (coffee extract); 4,632,832 to Okonogi et al. (instant creaming powder); 5,246,611, 5,571,782, 5,660,845 and 5,635,238 to Trinh et al. (perfumes, flavors, and pharmaceuticals); 4,548,811 to Kubo et al. (waving lotion); 6,287,603 to Prasad et al. (perfumes, flavors, and pharmaceuticals); 4,906,488 to Pera (olfactants, flavors, medicaments, and pesticides); and 6,638,557 to Qi et al. (fish oils).

Cyclodextrins are further described in the following publications, which are also incorporated herein by reference: (1) Reineccius, T. A., et al. “Encapsulation of Flavors Using Cyclodextrins: Comparison of Flavor Retention in Alpha, Beta, and Gamma Types.” Journal of Food Science. 2002; 67(9): 3271-3279; (2) Shiga, H., et al. “Flavor Encapsulation and Release Characteristics of Spray-Dried Powder by the Blended Encapsulant of Cyclodextrin and Gum Arabic.” Marcel Dekker, Inc., www.dekker.com. 2001; (3) Szente L., et al. “Molecular Encapsulation of Natural and Synthetic Coffee Flavor with β-cyclodextrin.” Journal of Food Science. 1986; 51(4): 1024-1027; (4) Reineccius, G. A., et al. “Encapsulation of Artificial Flavors by β-cyclodextrin.” Perfumer & Flavorist (ISSN 0272-2666) An Allured Publication. 1986: 11(4): 2-6; and (5) Bhandari, B. R., et al. “Encapsulation of Lemon Oil by Paste Method Using β-cyclodextrin: Encapsulation Efficiency and Profile of Oil Volatiles.” J. Agric. Food Chem. 1999; 47: 5194-5197.

### SUMMARY

The present invention provides a cyclodextrin inclusion complex comprising a guest encapsulated by cyclodextrin, the complex being greater than about 400 microns in size.

The present invention also provides a method of imparting flavor to a product to form a flavored product, the method comprising: incorporating a large particle cyclodextrin inclusion complex into a product to form a flavored product, the complex comprising a guest encapsulated by a cyclodextrin. The present invention further provides a flavored product comprising a large particle cyclodextrin inclusion complex.

The present invention also provides a method of making a large particle cyclodextrin inclusion complex comprising: (a) mixing cyclodextrin with solvent to form a first mixture; (b) adding a guest to the first mixture to form a second mixture; (c) adding a hardening agent to the second mixture to form a third mixture; and (d) drying the third mixture to form a large particle cyclodextrin inclusion complex.

### BRIEF DESCRIPTION OF THE DRAWINGS

FIG. 1 is a schematic illustration of a cyclodextrin molecule having a cavity, and a guest molecule held within the cavity.

FIG. 2 is a schematic illustration of a nano-structure formed by self-assembled cyclodextrin molecules and guest molecules.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

The present invention is generally directed to large particle cyclodextrin inclusion complexes and methods of forming them. Some large particle cyclodextrin inclusion complexes of the present invention provide for the encapsulation of volatile and reactive guest molecules. In some embodiments, the encapsulation of the guest molecule can provide at least one of the following: (1) prevention of a volatile or reactive guest from escaping a commercial product which may result in a lack of flavor intensity in the commercial product; (2) isolation of the guest molecule from interaction and reaction with other components that would cause off note formation; (3) stabilization of the guest molecule against degradation (e.g., hydrolysis, oxidation, etc.); (4) selective extraction of the guest molecule from other products or compounds; (5) enhancement of the water solubility of the guest molecule; (6) taste or odor improvement or enhancement of a commercial product; (7) thermal protection of the guest in a microwave and conventional baking applications; (8) slow and/or sustained release of flavor or odor; and (9) safe handling of guest molecules.

Some embodiments of the present invention provide a method for preparing a large particle cyclodextrin inclusion complex. The method can include blending cyclodextrin with a solvent such as water to form a first mixture, mixing a guest with the first mixture to form a second mixture, adding a hardening agent to the second mixture to form a third mixture and vacuum drying the third mixture.

In some embodiments of the present invention, a method for preparing a large particle cyclodextrin inclusion complex is provided. The method can include dry blending cyclodextrin and emulsifier and adding a solvent to the dry blend to form a first mixture, cooling the first mixture, adding a guest and mixing to form a second mixture, mixing a hardening agent with the second mixture to form a third mixture, and vacuum drying the third mixture.

Some embodiments of the present invention provide a large particle cyclodextrin inclusion complex including a guest molecule held within the cavity of the cyclodextrin. Suitably, a slight excess of cyclodextrin may be present.

As used herein, the term “cyclodextrin” can refer to a cyclic dextrin molecule that is formed by enzyme conversion of starch. Specific enzymes, e.g., various forms of cycloglycosyltransferase (CGTase), can break down helical structures that occur in starch to form specific cyclodextrin molecules having three-dimensional polyglucose rings with, e.g., 6, 7, or 8 glucose molecules. For example, α-CGTase can convert starch to α-cyclodextrin having 6 glucose units, β-CGTase can convert starch to β-cyclodextrin having 7 glucose units, and γ-CGTase can convert starch to γ-cyclodextrin having 8 glucose units. Cyclodextrins include, but are not limited to, at least one of α-cyclodextrin, β-cyclodextrin, γ-cyclodextrin, and combinations thereof. β-cyclodextrin is not known to have any toxic effects, is World-Wide GRAS (i.e., Generally Regarded As Safe) and natural, and is FDA approved. α-cyclodextrin and γ-cyclodextrin are also considered natural products and are U.S. and E.U. GRAS.

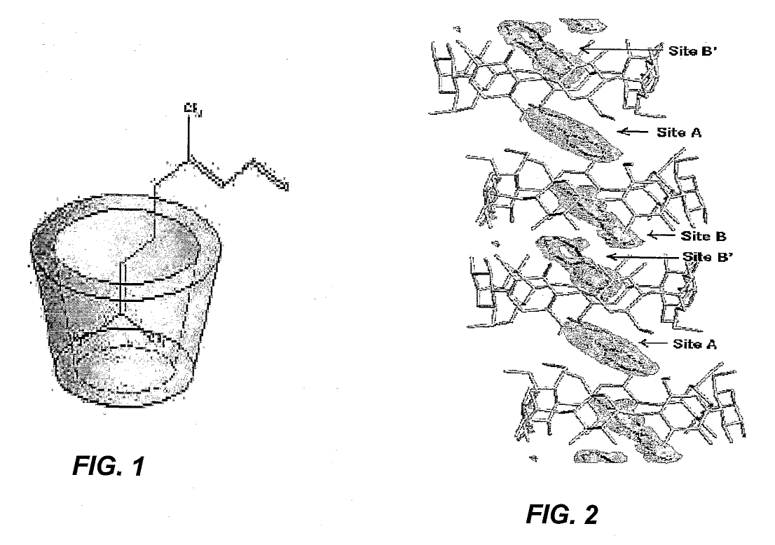

The three-dimensional cyclic structure (i.e., macrocyclic structure) of a cyclodextrin molecule 10 is shown schematically in FIG. 1. The cyclodextrin molecule 10 includes an external portion 12, which includes primary and secondary hydroxyl groups, and which is hydrophilic. The cyclodextrin molecule 10 also includes a three-dimensional cavity 14, which includes carbon atoms, hydrogen atoms and ether linkages, and which is hydrophobic. The hydrophobic cavity 14 of the cyclodextrin molecule can act as a host and hold a variety of molecules, or guests 16, that include a hydrophobic portion to form a large particle cyclodextrin inclusion complex.

As used herein, the term “guest” can refer to any molecule of which at least a portion can be held or captured within the three dimensional cavity present in the cyclodextrin molecule, including, without limitation, at least one of a flavor, an olfactant, a pharmaceutical agent, a nutraceutical agent (e.g., creatine), and combinations thereof.

Examples of flavors can include, without limitation, flavors based on aldehydes, ketones or alcohols. Examples of aldehyde flavors can include, without limitation, at least one of: acetaldehyde (apple); benzaldehyde (cherry, almond); anisic aldehyde (licorice, anise); cinnamic aldehyde (cinnamon); citral (e.g., geranial, alpha citral (lemon, lime) and neral, beta citral (lemon, lime); decanal (orange, lemon); ethyl vanillin (vanilla, cream); heliotropine, i.e. piperonal (vanilla, cream); vanillin (vanilla, cream); a-amyl cinnamaldehyde (spicy fruity flavors); butyraldehyde (butter, cheese); valeraldehyde (butter, cheese); citronellal (modifies, many types); decenal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2-6-dimethyl-5-heptenal, i.e. MELONAL™ (melon); 2,6-dimethyloctanal (green fruit); 2-dodecenal (citrus, mandarin); and combinations thereof.

Examples of ketone flavors can include, without limitation, at least one of: d-carvone (caraway); l-carvone (spearmint); diacetyl (butter, cheese, “cream”)); benzophenone (fruity and spicy flavors, vanilla); methyl ethyl ketone (berry fruits); maltol (berry fruits) menthone (mints), methyl amyl ketone, ethyl butyl ketone, dipropyl ketone, methyl hexyl ketone, ethyl amyl ketone (berry fruits, stone fruits); pyruvic acid (smokey, nutty flavors); acetanisole (hawthorn heliotrope); dihydrocarvone (spearmint); 2,4-dimethylacetophenone (peppermint); 1,3-diphenyl-2-propanone (almond); acetocumene (orris and basil, spicy); isojasmone (jasmine); d-isomethylionone (orris like, violet); isobutyl acetoacetate (brandy-like); zingerone (ginger); pulegone (peppermint-camphor); d-piperitone (minty); 2-nonanone (rose and tea-like); and combinations thereof.

Examples of alcohol flavors can include, without limitation, at least one of anisic alcohol or p-methoxybenzyl alcohol (fruity, peach); benzyl alcohol (fruity); carvacrol or 2-p-cymenol (pungent warm odor); carveol; cinnamyl alcohol (floral odor); citronellol (rose like); decanol; dihydrocarveol (spicy, peppery); tetrahydrogeraniol or 3,7-dimethyl-1-octanol (rose odor); eugenol (clove); p-mentha-1,8dien-7-Oλ or perillyl alcohol (floral-pine); alpha terpineol; mentha-1,5-dien-8-ol 1; mentha-1,5-dien-8-ol 2; p-cymen-8-ol; and combinations thereof.

Examples of olfactants can include, without limitation, at least one of natural fragrances, synthetic fragrances, synthetic essential oils, natural essential oils, and combinations thereof.

Examples of the synthetic fragrances can include, without limitation, at least one of terpenic hydrocarbons, esters, ethers, alcohols, aldehydes, phenols, ketones, acetals, oximes, and combinations thereof.

Examples of terpenic hydrocarbons can include, without limitation, at least one of lime terpene, lemon terpene, limonen dimer, and combinations thereof.

Examples of esters can include, without limitation, at least one of γ-undecalactone, ethyl methyl phenyl glycidate, allyl caproate, amyl salicylate, amyl benzoate, amyl acetate, benzyl acetate, benzyl benzoate, benzyl salicylate, benzyl propionate, butyl acetate, benzyl butyrate, benzyl phenylacetate, cedryl acetate, citronellyl acetate, citronellyl formate, p-cresyl acetate, 2-t-pentyl-cyclohexyl acetate, cyclohexyl acetate, cis-3-hexenyl acetate, cis-3-hexenyl salicylate, dimethylbenzyl acetate, diethyl phthalate, δ-deca-lactone dibutyl phthalate, ethyl butyrate, ethyl acetate, ethyl benzoate, fenchyl acetate, geranyl acetate, γ-dodecalatone, methyl dihydrojasmonate, isobornyl acetate, β-isopropoxyethyl salicylate, linalyl acetate, methyl benzoate, o-t-butylcylohexyl acetate, methyl salicylate, ethylene brassylate, ethylene dodecanoate, methyl phenyl acetate, phenylethyl isobutyrate, phenylethylphenyl acetate, phenylethyl acetate, methyl phenyl carbinyl acetate, 3,5,5-trimethylhexyl acetate, terpinyl acetate, triethyl citrate, p-t-butylcyclohexyl acetate, vetiver acetate, and combinations thereof.

Examples of ethers can include, without limitation, at least one of p-cresyl methyl ether, diphenyl ether, 1,3,4,6,7,8-hexahydro-4,6,7,8,8-hexamethyl cyclopenta-O-2-benzopyran, phenyl isoamyl ether, and combinations thereof.

Examples of alcohols can include, without limitation, at least one of n-octyl alcohol, n-nonyl alcohol, β-phenylethyldimethyl carbinol, dimethyl benzyl carbinol, carbitol dihydromyrcenol, dimethyl octanol, hexylene glycol linalool, leaf alcohol, nerol, phenoxyethanol, γ-phenyl-propyl alcohol, β-phenylethyl alcohol, methylphenyl carbinol, terpineol, tetraphydroalloocimenol, tetrahydrolinalool, 9-decen-1-ol, and combinations thereof.

Examples of aldehydes can include, without limitation, at least one of n-nonyl aldehyde, undecylene aldehyde, methylnonyl acetaldehyde, anisaldehyde, benzaldehyde, cyclamenaldehyde, 2-hexylhexanal, ahexylcinnamic alehyde, phenyl acetaldehyde, 4-(4-hydroxy-4-methylpentyl)-3-cyclohexene-1-carboxyaldehyde, p-t-butyl-a-methylhydro-cinnamic aldehyde, hydroxycitronellal, α-amylcinnamic aldehyde, 3,5-dimethyl-3-cyclohexene-1-carboxyaldehyde, and combinations thereof.

Examples of phenols can include, without limitation, methyl eugenol.

Examples of ketones can include, without limitation, at least one of 1-carvone, α-damascone, ionone, 4-t-pentylcyclohexanone, 3-amyl-4-acetoxytetrahydropyran, menthone, methylionone, p-t-amycyclohexanone, acetyl cedrene, and combinations thereof.

Examples of the acetals can include, without limitation, phenylacetaldehydedimethyl acetal.

Examples of oximes can include, without limitation, 5-methyl-3-heptanon oxime.

A guest can further include, without limitation, at least one of fatty acids, fatty acid triglcerides, omega-3-fatty acids and triglycerides thereof, tocopherols, lactones, terpenes, diacetyl, dimethyl sulfide, proline, furaneol, linalool, acetyl propionyl, cocoa products, natural essences (e.g., orange, tomato, apple, cinnamon, raspberry, etc.), essential oils (e.g., orange, lemon, lime, etc.), sweeteners (e.g., aspartame, neotame, acesulfame-K, saccharin, neohesperidin dihydrochalcone, glycyrrhiza, and stevia derived sweeteners), sabinene, p-cymene, p,a-dimethyl styrene, and combinations thereof.

As used herein, the term “log(P)” or “log(P) value” is a property of a material that can be found in standard reference tables, and which refers to the material's octanol/water partition coefficient. Generally, the log(P) value of a material is a representation of its hydrophilicity/hydrophobicity. P is defined as the ratio of the concentration of the material in octanol to the concentration of the material in water. Accordingly, the log(P) of a material of interest will be negative if the concentration of the material in water is higher than the concentration of the material in octanol. The log (P) value will be positive if the concentration is higher in octanol, and the log(P) value will be zero if the concentration of the material of interest is the same in water as in octanol. Accordingly, guests can be characterized by their log(P) value. For reference, Table 1A lists log(P) values for a variety of materials, some of which may be guests of the present invention.

TABLE 1A
Log (P) values for a variety of guests

| Material | CAS# | log P¹ | molecular wt |
|---|---|---|---|
| Creatine | 57-00-1 | −3.72 | 131 |
| Praline | 147-85-3 | −2.15 | 115 |
| Diacetyl | 431-03-8 | −1.34 | 86 |
| Methanol | 67-56-1 | −0.74 | 32 |
| Ethanol | 64-17-5 | −0.30 | 46 |
| Acetone | 67-64-1 | −0.24 | 58 |
| Maltol | 118-71-8 | −0.19 | 126 |
| ethyl lactate | 97-64-3 | −0.18 | 118 |
| acetic acid | 64-19-7 | −0.17 | 60 |
| acetaldehyde | 75-07-0 | −0.17 | 44 |
| Aspartame | 22839-47-0 | 0.07 | 294 |
| ethyl levulinate | 539-88-8 | 0.29 | 144 |
| ethyl maltol | 4940-11-8 | 0.30 | 140 |
| Furaneol | 3658-77-3 | 0.82 | 128 |
| dimethyl sulfide | 75-18-3 | 0.92 | 62 |
| vanillin | 121-33-5 | 1.05 | 152 |
| benzyl alcohol | 100-51-6 | 1.05 | 108 |
| raspberry ketone | 5471-51-2 | 1.48 | 164 |
| benzaldehyde | 100-52-7 | 1.48 | 106 |
| ethyl vanillin | 121-32-4 | 1.50 | 166 |
| phenethyl alcohol | 60-12-8 | 1.57 | 122 |
| cis-3-hexenol | 928-96-1 | 1.61 | 100 |
| trans-2-hexenol | 928-95-0 | 1.61 | 100 |
| whiskey fusel oils | mixture | 1.75 | 74 |
| ethyl isobutyrate | 97-62-1 | 1.77 | 116 |
| ethyl butyrate | 105-54-4 | 1.85 | 116 |
| hexanol | 111-27-3 | 2.03 | 102 |
| ethyl-2-methyl butyrate | 7452-79-1 | 2.26 | 130 |
| ethyl isovalerate | 108-64-5 | 2.26 | 130 |
| isoamyl acetate | 123-92-2 | 2.26 | 130 |
| nutmeg oil | mixture | 2.90 | 164 |
| methyl isoeugenol | 93-16-3 | 2.95 | 164 |
| gamma undecalactone | 104-67-6 | 3.06 | 184 |
| alpha terpineol | 98-55-5 | 3.33 | 154 |
| chlorocyclohexane (CCH) | 542-18-7 | 3.36 | 118 |
| linalool | 78-70-6 | 3.38 | 154 |
| citral | 5392-40-5 | 3.45 | 152 |
| geraniol | 106-24-1 | 3.47 | 154 |
| citronellol | 106-22-9 | 3.56 | 154 |
| p-cymene | 99-87-6 | 4.10 | 134 |
| limonene | 138-86-3 | 4.83 | 136 |

Examples of guests having a relatively large positive log(P) value (e.g., greater than about 2) include, but are not limited to, citral, linalool, alpha terpineol, and combinations thereof. Examples of guests having a relatively small positive log(P) value (e.g. less than about 1 but greater than zero) include, but are not limited to, dimethyl sulfide, furaneol, ethyl maltol, aspartame, and combinations thereof. Examples of guests having a relatively large negative log(P) value (e.g., less than about −2) include, but are not limited to, creatine, proline, and combinations thereof. Examples of guests having a relatively small negative log(P) value (e.g., less than 0 but greater than about −2) include, but are not limited to, diacetyl, acetaldehyde, maltol, and combinations thereof.

Log(P) values are significant in many aspects of food and flavor chemistry. A table of log(P) values is provided above. The log(P) values of guests can be important to many aspects of an end product (e.g., foods and flavors). Generally, organic guest molecules having a positive log(P) can be successfully encapsulated in cyclodextrin. In a mixture comprising several guests, competition can exist, and log(P) values can be useful in determining which guests will be more likely to be successfully encapsulated. Maltol and furaneol are examples of two guests that have similar flavor characteristics (i.e., sweet attributes), but which would have different levels of success in cyclodextrin encapsulation because of their differing log(P) values. Log(P) values may be important in food products with a high aqueous content or environment. Compounds with significant and positive log(P) values are, by definition, the least soluble and therefore the first to migrate, separate, and then be exposed to change in the package. The high log(P) value, however, may make them effectively scavenged and protected by addition cyclodextrin in the product.

As mentioned above, the cyclodextrin used with the present invention can include α-cyclodextrin, β-cyclodextrin, γ-cyclodextrin, and combinations thereof. Suitably, the cyclodextrin may be derivatized, with e.g., hydroxypropyl groups. In embodiments in which a more hydrophilic guest (i.e., having a smaller log(P) value) is used, α-cyclodextrin may be used (i.e., alone or in combination with another type of cyclodextrin) to improve the encapsulation of the guest in cyclodextrin. For example, a combination of α-cyclodextrin and β-cyclodextrin can be used in embodiments employing relatively hydrophilic guests to improve the formation of a large particle cyclodextrin inclusion complex.

As used herein, the term “cyclodextrin inclusion complex” refers to a complex that is formed by encapsulating at least a portion of one or more guest molecules with one or more cyclodextrin molecules (encapsulation on a molecular level) by capturing and holding a guest molecule within the three dimensional cavity. The guest can be held in position by van der Waal forces within the cavity by at least one of hydrogen bonding and hydrophilic-hydrophobic interactions. The guest can be released from the cavity when the cyclodextrin inclusion complex is dissolved in water. Cyclodextrin inclusion complexes are also referred to herein as “guest-cyclodextrin complexes.” Because the cavity of cyclodextrin is hydrophobic relative to its exterior, guests having positive log(P) values (particularly, relatively large positive log(P) values) will encapsulate easily in cyclodextrin and form stable cyclodextrin inclusion complexes in an aqueous environment, because the guest will thermodynamically prefer the cyclodextrin cavity to the aqueous environment. In some embodiments, when it is desired to complex more than one guest, each guest can be encapsulated separately to maximize the efficiency of encapsulating the guest of interest. In some embodiments, the use of a solvent with a significant positive log(P) value, such as benzyl alcohol or limonene, enhances the complexation and stabilization of a wide range of guests in large particle cyclodextrin inclusion complexes. Suitably, the cyclodextrin inclusion complex has a guest to cyclodextrin ratio of about 0.2:1 to about 2:1. In an alternative embodiment, the guest to cyclodextrin ratio is about 0.5:1 to about 1:1.

As used herein, the term “large particle cyclodextrin inclusion complex” generally refers to a cyclodextrin inclusion complex that is greater than about 400 microns in size. Suitably, the cyclodextrin inclusion complex is greater than about 500 microns, about 600 microns, about 700 microns or about 800 microns. For certain embodiments, the cyclodextrin inclusion complexes of the present invention are about 850 to about 1000 microns in size. For other embodiments, the cyclodextrin inclusion complexes are about 400 to about 1000 microns in size, or about 500 to about 800 microns, or about 600 to about 700 microns. The large particle cyclodextrin inclusion complexes of the present invention are about 2 times bigger than the equivalent spray dry version of the cyclodextrin inclusion complex (which is about 177 microns or smaller), or about 3 times as big, or about 5 times as big, or about 10 times as big, or about 20 times as big, or about 50 times as big, or about 70 times as big, or about 90 times as big, or about 100 times as big. The complexes of the present invention can be milled or ground to any size without sacrificing stability or leakage of liquid material.

As used herein, the term “hydrocolloid” generally refers to a substance that forms a gel with water. A hydrocolloid can include, without limitation, at least one of xanthan gum, pectin, gum arabic (or gum acacia), tragacanth, guar, carrageenan, locust bean, and combinations thereof.

As used herein, the term “pectin” refers to a hydrocolloidal polysaccharide that can occur in plant tissues (e.g., in ripe fruits and vegetables). Pectin can include, without limitation, at least one of beet pectin, fruit pectin (e.g., from citrus peels), and combinations thereof. The pectin employed can be of varying molecular weight.

As used herein, the term “hardening agent” generally refers to a substance that aids in the formation of hard crystals of the cyclodextrin inclusion complex. A hardening agent can include, without limitation, at least one of sucrose, other sugars, gum acacia, gum acacia substitutes such as dextrose, modified food starch (e.g. EmCap® sold by Cargill), and corn syrup solids, carboxymethylcellulose, citric acid, sorbitol, and combinations thereof. The hardening agent can add numerous adaptive features such as color, acidity, controlled solubility etc Suitably, the hardening agent is present in about 5% to about 35% by weight of the total weight of cyclodextrin, solvent and guest. In another embodiment, the hardening agent is present in about 10% to about 25% by weight of the total weight of cyclodextrin, solvent and guest. In yet another embodiment, the hardening agent is present in about 10% to about 15% by weight of the total weight of cyclodextrin, solvent and guest.

Large particle cyclodextrin inclusion complexes of the present invention can be used in a variety of applications or end products, including, without limitation, at least one of foods (e.g., beverages, soft drinks, salad dressings, popcorn, cereal, coffee, tea, cookies, brownies, other desserts, other baked goods, seasonings, etc.), chewing gums, dentifrices, such as toothpaste and mouth rinses, candy, flavorings, fragrances, pharmaceuticals, nutraceuticals, cosmetics, agricultural applications (e.g., herbicides, pesticides, etc.), photographic emulsions, laundry detergents and combinations thereof. In some embodiments, cyclodextrin inclusion complexes can be used as intermediate isolation matrices to be further processed, isolated and dried (e.g., as used with waste streams).

Large particle cyclodextrin inclusion complexes are particularly well suited for use in tea bags, french fries, breadings (e.g. for onion rings, chicken patties, fish patties, and the like), batter, pizza crust and dough (e.g. to prevent the garlic and onion flavors from affecting rising of the dough), and in pizza sauce. The large particle cyclodextrin inclusion complexes of the present invention may also be used in controlled release applications such as fry coatings and baking mixes or for topical application to cereals and snacks, where visual particles are desired or where non-linear flavor delivery (e.g. for bursts of flavor) is desired or where sequential delivery (e.g. changing color or profile based on temperature, pH, or moisture) is desired. The large particle cyclodextrin complexes may also be used in gourmet cooking ingredients (e.g. for wine and sherry). In addition, large particle cyclodextrin complexes can be used to mask the bitter taste of dentifrices containing active ingredients such as stannous fluoride, sodium hexametaphosphate and cetylpyridinium chloride, such as the CREST® PRO-HEALTH® toothpaste and mouth rinses, which are described in U.S. Pat. Nos. 6,696,045 and 6,740,311 each of which is fully incorporated by reference herein. For example, the large particle cyclodextrin complexes of the present invention can be used in dentifrices which protect against one or more of the following conditions: cavities, gingivitis, plaque, sensitive teeth, tartar buildup, stains, and bad breath. Suitably, the dentifrice contains little or no alcohol.

Suitably, the large particle cyclodextrin inclusion complex is present in an amount from about 0.001% to about 5% by weight. In another embodiment, the large particle cyclodextrin inclusion complex is present in an amount from about 0.01% to about 3% by weight. In yet another embodiment, the large particle cyclodextrin inclusion complex is present in an amount from about 0.1% to about 2% by weight of the product. In dentifrice applications, the large particle cyclodextrin inclusion complex may be present in about 0.01% to about 2% by weight of the product. In beverage applications, the large particle cyclodextrin inclusion complex may be present in an amount from about 0.01% to about 1.0% by weight of the product.

Large particle cyclodextrin inclusion complexes can be used to enhance the stability of the guest, or otherwise modify its solubility, delivery or performance. The amount of the guest molecule that can be encapsulated is directly related to the molecular weight of the guest molecule. In some embodiments, one mole of cyclodextrin encapsulates one mole of guest. According to this mole ratio, and by way of example only, in embodiments employing diacetyl (molecular weight of 86 Daltons) as the guest, and β-cyclodextrin (molecular weight 1135 Daltons), the maximum theoretical retention is (86/(86+1135))×100=7.04 wt %.

Cyclodextrin inclusion complexes form in solution. The drying process temporarily locks at least a portion of the guest in the cavity of the cyclodextrin and can produce dry large particles of the cyclodextrin inclusion complex.

The hydrophobic (water insoluble) nature of the cyclodextrin cavity will preferentially trap like (hydrophobic) guests most easily at the expense of more water-soluble (hydrophilic) guests. This phenomenon can result in an imbalance of components as compared to typical spray drying and a poor overall yield.

In some embodiments of the present invention, the competition between hydrophilic and hydrophobic effects is avoided by selecting key ingredients to encapsulate separately. For example, in the case of butter flavors, fatty acids and lactones form cyclodextrin inclusion complexes more easily than diacetyl. However, these compounds are not the key character impact compounds associated with butter, and they will reduce the overall yield of diacetyl and other water soluble and volatile ingredients. In some embodiments, the key ingredient in butter flavor (i.e., diacetyl) is maximized to produce a high impact, more stable, and more economical product. By way of further example, in the case of lemon flavors, most lemon flavor components will encapsulate equally well in cyclodextrin. However, terpenes (a component of lemon flavor) have little flavor value, and yet make up approximately 90% of a lemon flavor mixture, whereas citral is a key flavor ingredient for lemon flavor. In some embodiments, citral is encapsulated alone. By selecting key ingredients (e.g., diacetyl, citral, etc.) to encapsulate separately, the complexity of the starting material is reduced, allowing optimization of engineering steps and process economics.

In some embodiments, the viscosity of the suspension, emulsion or mixture formed by mixing the cyclodextrin and guest molecules in a solvent is controlled. An emulsifier (e.g., a thickener, gelling agent, polysaccharide, hydrocolloid) can be added to maintain intimate contact between the cyclodextrin and the guest, and to aid in the inclusion process. Particularly, low molecular weight hydrocolloids can be used. One preferred hydrocolloid is pectin. Emulsifiers can aid in the inclusion process without requiring the use of high heat or co-solvents (e.g., ethanol, acetone, isopropanol, etc.) to increase solubility.

In some embodiments, the moisture content of the suspension, emulsion or mixture is reduced to essentially force the guest to behave as a hydrophobic compound. This process can increase the retention of even relatively hydrophilic guests, such as acetaldehyde, diacetyl, dimethyl sulfide, etc.

In some embodiments of the present invention, a large particle cyclodextrin inclusion complex can be formed by the following paste process, which may include some or all of the following steps:

(1) Blending cyclodextrin with a solvent (e.g. water and/or ethanol) to form a paste (e.g., for about 20 minutes to 2 hours);

(2) Adding a guest and stirring (e.g., for approximately 0.5 minutes to 4 hours);

(3) Adding a hardening agent and stirring until uniform (e.g., for approximately 15 minutes); and

(4) Vacuum drying the cyclodextrin inclusion complex; and

(5) Grinding or milling the dry cyclodextrin inclusion complex to form large particles.

These steps need not necessarily be performed in the order listed. In addition, the above paste process has proved to be very robust in that the process can be performed using variations in temperature, time of mixing, and other process parameters. Suitably, the solvent is a water miscible solvent. For example, the solvent may be water or a lower alcohol, e.g. ethanol or isopropanol, propylene glycol or glycerin.

A color agent may be added during step 3 of the above process.

If the particles resulting from step 5 are not of sufficient size, they can be rewet and vacuum dried again to form larger particles. The ability to rewet and recycle the particles allows for up to about 100% utilization of the cyclodextrin inclusion complex.

The blending in step 1 and the stirring in step 3 and 4 can be accomplished by at least one of shaking, stirring, tumbling, and combinations thereof.

Steps 1 to 3 in the paste process described above can be accomplished in a reactor that is jacketed for heating, cooling, or both. In some embodiments, the combining and agitating can be performed at room temperature. In some embodiments, the combining and agitating can be performed at a temperature greater than room temperature. The reactor size can be dependent on the production size. For example, a 100 gallon reactor can be used. The reactor can include a paddle agitator and a condenser unit. In some embodiments, step 1 is completed in the reactor, and in step 2, hot deionized water is added to the dry blend of cyclodextrin and emulsifier in the same reactor.

In other embodiments of the present invention, a large particle cyclodextrin inclusion complex can be formed by the following dry blending process, which may include some or all of the following steps:

(1) Dry blending cyclodextrin and an emulsifier (e.g., pectin);

(2) Combining the dry blend of cyclodextrin and the emulsifier with a solvent such as water in a reactor, and agitating;

(3) Cooling the reactor (e.g., turning on a cooling jacket);

(4) Adding the guest and stirring (e.g., for approximately 5 to 8 hours);

(5) Adding a hardening agent and stirring;

(6) Vacuum drying the cyclodextrin inclusion complex; and

(7) Grinding or milling the dry cyclodextrin inclusion complex to form large particles.

These steps need not necessarily be performed in the order listed. In addition, the above dry blending process has proved to be very robust in that the process can be performed using variations in temperature, time of mixing, and other process parameters. Suitably, the solvent is a water miscible solvent. For example, the solvent may be water or a lower alcohol, e.g. ethanol or isopropanol, propylene glycol or glycerin.

If the particles resulting from step 7 are not of sufficient size, they can be rewet and vacuum dried again to form larger particles.

In some embodiments, step 1 in the process described above can be accomplished using an in-tank mixer in the reactor to which the hot water will be added in step 2. For example, in some embodiments, the process above is accomplished using a 1000 gallon reactor equipped with a jacket for temperature control and an inline high shear mixer. In some embodiments, the cyclodextrin and emulsifier can be dry blended in a separate apparatus (e.g., a ribbon blender, etc.) and then added to the reactor in which the remainder of the above process is completed.

A variety of weight percentages of an emulsifier to cyclodextrin can be used, including, without limitation, an emulsifier:cyclodextrin weight percentage of at least about 0.5%, particularly, at least about 1%, and more particularly, at least about 2%. In addition, an emulsifier:cyclodextrin weight percentage of less than about 10% can be used, particularly, less than about 6%, and more particularly, less than about 4%.

Step 2 in the process described above can be accomplished in a reactor that is jacketed for heating, cooling, or both. In some embodiments, the combining and agitating can be performed at room temperature. In some embodiments, the combining and agitating can be performed at a temperature greater than room temperature. The reactor size can be dependent on the production size. For example, a 100 gallon reactor can be used. The reactor can include a paddle agitator and a condenser unit. In some embodiments, step 1 is completed in the reactor, and in step 2, hot deionized water is added to the dry blend of cyclodextrin and emulsifier in the same reactor.

Step 3 can be accomplished using a coolant system that includes a cooling jacket. For example, the reactor can be cooled with a propylene glycol coolant and a cooling jacket.

Step 4 can be accomplished in a sealed reactor, or the reactor can be temporarily exposed to the environment while the guest is added, and the reactor can be re-sealed after the addition of the guest. Heat can be added when the guest is added and during the stirring of step 4. For example, in some embodiments, the mixture is heated to about 50-60° C.

The agitating in step 2, the stirring in step 4, and the stirring in step 5 can be accomplished by at least one of shaking, stirring, tumbling, and combinations thereof.

The processes outlined above can be used to provide large particle cyclodextrin inclusion complexes with a variety of guests for a variety of applications or end products. For example, some of the embodiments of the present invention provide a large particle cyclodextrin inclusion complex with a guest comprising lemon oil, which can be used for various food products as a lemon flavoring (e.g., in tea, etc.).

A dramatic improvement in physical durability, complexation rate, and controlled solubility and release was unexpectedly found when the ratio of solvent to cyclodextrin was reduced. It also should be noted that improved processing can be achieved by removing the majority of water from the reaction mixture by, e.g. decanting, settling or centrifugation. The hardening agents can be added pre- or post-water removal. Suitably, the cyclodextrin to solvent ratio may be from about 30:70 to about 70:30. In another embodiment, the ratio may be from about 45:55 to about 65:35. In yet another embodiment, the ratio may be from about 50:50 to about 60:40.

A general point, known to those skilled in the art, concerns the end point of drying. The paste or wet inclusion complex, when placed in a vacuum oven will cool until the moisture level drops below approximately 4%. In practice, as vacuum is applied to trays of inclusion complex, the temperature of the tray contents will drop for the duration of the drying process, elevating on complete moisture removal. In the examples, the oven is set to 79° C. with an applied vacuum of 1 millitorr. As solvent is removed, the temperature of the product will fall to approximately 0-10° C. The end point is determined by the temperature of the dried paste returning to the oven temperature of 79° C.

The encapsulation of the guest molecule can provide isolation of the guest molecule from interaction and reaction with other components that would cause off note formation and stabilization of the guest molecule against degradation (e.g., hydrolysis, oxidation, etc.). Stabilization of the guest against degradation can improve or enhance the desired effect or function (e.g., taste, odor, etc.) of a resulting commercial product that includes the encapsulated guest.

Many guests can degrade and create off-notes that can detract from a main or desired effect or function. For example, many flavors or olfactants can degrade and create off-note flavors or odors that can detract from the desired flavor or odor of a commercial product. Guests can also be degraded by means of photo-oxidation. The rate of degradation of the guest (i.e., the rate of formation of off-note(s)) is generally governed by the following generic kinetic rate equation:

$$Rate \approx \frac{{[ offnote ]}^{z}}{{[ guest ]}^{x} \cdot {[ RC ]}^{y}}$$

where [guest] refers to the molar concentration of guest in a solution, [RC] refers to the molar concentration of a reactive compound in a solution responsible for reacting with and degrading the guest (e.g., an acid), and [offnote] refers to the molar concentration of off-notes formed. The powers x, y and z represent kinetic order, depending on the reaction that occurs between a guest of interest and the corresponding reactive compound(s) present in solution to produce off-notes. Thus, the rate of degradation of the guest is proportional to the product of the molar concentrations of the guest and any reactive compounds, raised to a power determined by the kinetic order of the reaction.

Any of the above-mentioned guests can be protected and stabilized in this manner. For example, cyclodextrin can be used to protect and/or stabilize a variety of guest molecules to enhance the desired effect or function of a product, including, but not limited to, the following guest molecules: citral, benzaldehyde, alpha terpineol, vanillin, aspartame, neotame, acetaldehyde, creatine, and combinations thereof.

Citral (log(P)=3.45) is a citrus or lemon flavor that can be used in various applications, such as acidic beverages. Acidic beverages can include, but are not limited to lemonade, 7UP® lemon-lime flavored soft drink (registered trademark of Dr. Pepper/Seven-Up, Inc.), SPRITE® lemon-lime flavored soft drink (registered trademark of The Coca-Cola Company, Atlanta, Ga.), SIERRA MIST® lemon-lime flavored soft drink (registered trademark of Pepsico, Purchase, N.Y.), tea (e.g., LIPTON® and BRISK®, registered trademarks of Lipton), alcoholic beverages, and combinations thereof. Alpha terpineol (log(P)=3.33) is a lime flavor that can be used in similar products as those listed above with respect to citral.

Benzaldehyde (log(P)=1.48) is a cherry flavor that can be used in a variety of applications, including acidic beverages. An example of an acidic beverage that can be flavored with benzaldehyde includes, but is not limited to CHERRY COKE® cherry-cola flavored soft drink (registered trademark of The Coca-Cola Company, Atlanta, Ga.).

Vanillin (log(P)=1.05) is a vanilla flavor that can be used in a variety of applications, including, but not limited to, vanilla-flavored beverages, baked goods, etc., and combinations thereof.

Aspartame (log(P)=0.07) is a non-sucrose sweetener that can be used in a variety of diet foods and beverages, including, but not limited to, diet soft drinks. Neotame is also a non-sucrose sweetener that can be used in diet foods and beverages.

Acetaldehyde (log(P)=−0.17) is an apple flavor that can be used in a variety of applications, including, but not limited to, foods, beverages, candies, etc., and combinations thereof.

Creatine (log(P)=−3.72) is a nutraceutical agent that can be used in a variety of applications, including, but not limited to, nutraceutical formulations. Examples of nutraceutical formulations include, but are not limited to, powder formulations that can be combined with milk, water or another liquid, and combinations thereof.

The formation of the cyclodextrin inclusion complex in solution between the guest and the cyclodextrin can be more completely represented by the following equation:

$${S}_{( aq )} + {CD}_{( aq )} {}^{{K}_{P2}} S \cdot {CD}_{( aq )} ; {K}_{P2} = \frac{{[ S \cdot CD ]}_{( aq )}}{{{[ S ]}_{( aq )} [ CD ]}_{( aq )}} \qquad ( 9 )$$

The log(P) value of the guest can be a factor in the formation and stability of the cyclodextrin inclusion complex. That is, it has been shown that the equilibrium shown in equation 9 above is driven to the right by the net energy loss accompanied by the encapsulation process in solution, and that the equilibrium can be at least partially predicted by the log(P) value of the guest of interest. It has been found that log(P) values of the guests can be a factor in end products with a high aqueous content or environment. For example, guests with relatively large positive log(P) values are typically the least water-soluble and can migrate and separate from an end product, and can be susceptible to a change in the environment within a package. However, the relatively large log(P) value can make such guests effectively scavenged and protected by the addition of cyclodextrin to the end product. In other words, in some embodiments, the guests that have traditionally been the most difficult to stabilize can be easy to stabilize using the methods of the present invention.

To account for the effect of the log(P) value of the guest, the equilibrium constant (K_{P2}′) that represents the stability of the guest in a system can be represented by the following equation:

$${K}_{P2}^{′} = log ( P ) \frac{{[ S \cdot CD ]}_{( aq )}}{{{[ S ]}_{( aq )} [ CD ]}_{( aq )}} \qquad ( 10 )$$

wherein log(P) is the log(P) value for the guest (S) of interest in the system. Equation 10 establishes a model that takes into account a guest's log(P) value. Equation 10 shows how a thermodynamically stable system can result from first forming a cyclodextrin inclusion complex with a guest having a relatively large positive log(P) value. For example, in some embodiments, a stable system (i.e., a guest stabilizing system) can be formed using a guest having a positive log(P) value. In some embodiments, a stable system can be formed using a guest having a log(P) value of at least about +1. In some embodiments, a stable system can be formed using a guest having a log(P) value of at least about +2. In some embodiments, a stable system can be formed using a guest having a log(P) value of at least about +3. In the case of the large particle cyclodextrin complexes of the present invention, K_{P2}′ can be considered a major stabilizing effect, especially in toothpastes, fillings, coatings etc., where water activity (a_{w}) is low.

While log(P) values can be good empirical indicators and are available from several references, another important criteria is the binding constant for a particular guest (i.e., once a complex forms, how strongly is the guest bound in the cyclodextrin cavity). Unfortunately, the binding constant for a guest is determined experimentally. In the case of limonene and citral, for example, citral can form a much stronger complex, even though the log(P) values are similar. As a result, even in the presence of high limonene concentrations, citral is preferentially protected until consumption, because of its higher binding constant. This is an unexpected benefit and is not directly predicted from the current scientific literature.

In some embodiments, the cyclodextrin is added to the system in a molar ratio of cyclodextrin:guest of greater than 1:1. As shown in equation 10, stabilization of the guest in the system by cyclodextrin can be predicted by the log(P) value of the guest. In some embodiments, the guest chosen has a positive log(P) value. In some embodiments, the guest has a log(P) value of greater than about +1. In some embodiments, the guest has a log(P) value of greater than about +2. In some embodiments, the guest has a log(P) value of greater than about +3.

By taking into account the log(P) of the guest, it is possible to predict the stability of the guest in a system that comprises cyclodextrin. By exploiting the thermodynamics of the complexation in solution, a protective and stable environment can be formed for the guest, and this can be driven further by the addition of excess uncomplexed cyclodextrin. Release characteristics of a guest from the cylodextrin can be governed by K_{H}, the guest's air/water partition coefficient. K_{H} can be large compared to log(P) if the system comprising the cyclodextrin inclusion complex is placed in a non-equilibrium situation, such as the mouth. One of ordinary skill in the art will understand that more than one guest can be present in a system, and that similar equations and relationships can be applied to each guest of the system.

In addition, the use of the hardening agent in the method of the present invention pulls water from the paste helping to shift the equilibrium toward complexation. Crystal formation may be thermodynamically favored.

Various features and aspects of the invention are set forth in the following examples, which are intended to be illustrative and not limiting. All of the examples were performed at atmospheric pressure, and room temperature and all cyclodextrins were purchased from WACKER SPECIALITIES (Wacker Chemical Corp., Adrian, Mich.) unless stated otherwise.

### Example 1

### Formation of Large Particle Cyclodextrin Inclusion Complexes with Blueberry Flavor and 8% Sucrose

At atmospheric pressure, in a 2 liter reactor, 400.0000 g of β-cyclodextrin was dry blended with 8.00 g of beet pectin (2.0 wt % of pectin: β-cyclodextrin; XPQ EMP 4 beet pectin available from Degussa-France) to form a dry blend. The reactor was jacketed for heating and cooling, included a paddle agitator, and included a condenser unit. The reactor was supplied with a propylene glycol coolant at approximately 40° F. (4.5° C.). The propylene glycol coolant system was initially turned off, and the jacket acted somewhat as an insulator for the reactor. 1000.0000 g of deionized water was added to the dry blend of β-cyclodextrin and pectin. The mixture was stirred for approximately 1 hour using the paddle agitator of the reactor. The reactor was then temporarily opened, and 12.5000 g of blueberry flavor (Cargill Flavor Systems, 030-02212) was added. The reactor was resealed, the heating system was turn on to 50° C. and the resulting mixture was stirred overnight. The mixture was chilled to 10° C. and stirred for 2 additional hours. 32.0 g (8% of the cyclodextrin weight) of sucrose was added. Stirring continued for an additional hour. The mixture was then vacuum dried at 79° C. for 12 hours in a Heraeus Instruments vacutherm unit. The vacuum read approximately 1 mbar.

A percent retention of 3 wt % of blueberry flavor in the cyclodextrin inclusion complex was achieved. The moisture content was measured at 4%. The cyclodextrin inclusion complex included less than 0.05% surface blueberry flavor, and the particle size of the cyclodextrin inclusion complex was measured as 95% through a 10 mesh screen or 1500 microns, with greater than 60% holding on a 20 mesh screen (840 microns). Thus, the particle size was considered to be between 10 mesh (1500 microns) and 20 mesh (approximately 850 microns). Those skilled in the art will understand that heating and cooling can be controlled by other means.

### Example 2

### Formation of Large Particle Cyclodextrin Inclusion Complexes with Blueberry Flavor and 10% Gum Acacia

At atmospheric pressure, in a 2 liter reactor, 400.0000 g of β-cyclodextrin was dry blended with 8.00 g of beet pectin (2.0 wt % of pectin: β-cyclodextrin; XPQ EMP 4 beet pectin available from Degussa-France) to form a dry blend. The reactor was jacketed for heating and cooling, included a paddle agitator, and included a condenser unit. The reactor was supplied with a propylene glycol coolant at approximately 40° F. (4.5° C.). The propylene glycol coolant system was initially turned off, and the jacket acted somewhat as an insulator for the reactor. 1000.0000 g of deionized water was added to the dry blend of β-cyclodextrin and pectin. The mixture was stirred for approximately 1 hour using the paddle agitator of the reactor. The reactor was then temporarily opened, and 12.5000 g of blueberry flavor (Cargill Flavor Systems, 030-02212) was added. The reactor was resealed, the heating system was turn on to 50° C. and the mixture was stirred overnight. The mixture was chilled to 10° C. and stirred for 2 additional hours. 40.0 g (10% of the cyclodextrin weight) of gum acacia was added. Stirring continued for an additional hour. The mixture was then vacuum dried at 79° C. for 12 hours in a Heraeus Instruments vacutherm unit. The vacuum read approximately 1 mbar.

A percent retention of 3 wt % of blueberry flavor in the cyclodextrin inclusion complex was achieved. The moisture content was measured at 4%. The cyclodextrin inclusion complex included less than 0.05% surface blueberry flavor, and the particle size of the cyclodextrin inclusion complex was measured as 95% through a 10 mesh screen or 1500 microns, with greater than 50% holding on a 20 mesh screen (840 microns). Thus, the particle size was considered to be between 10 mesh (1500 microns) and 20 mesh (approximately 850 microns). Those skilled in the art will understand that heating and cooling can be controlled by other means.

### Example 3

### Formation of Large Particle Cyclodextrin Inclusion Complexes with Blueberry Flavor and 15% Gum Acacia

At atmospheric pressure, in a 2 liter reactor, 400.0000 g of β-cyclodextrin was dry blended with 8.00 g of beet pectin (2.0 wt % of pectin: β-cyclodextrin; XPQ EMP 4 beet pectin available from Degussa-France) to form a dry blend. The reactor was jacketed for heating and cooling, included a paddle agitator, and included a condenser unit. The reactor was supplied with a propylene glycol coolant at approximately 40° F. (4.5° C.). The propylene glycol coolant system was initially turned off, and the jacket acted somewhat as an insulator for the reactor. 1000.0000 g of deionized water was added to the dry blend of β-cyclodextrin and pectin. The mixture was stirred for approximately 1 hour using the paddle agitator of the reactor. The reactor was then temporarily opened, and 12.5000 g of blueberry flavor (Cargill Flavor Systems, 030-02212) was added. The reactor was resealed, the heating system was turn on to 50° C. and the mixture was stirred overnight. The mixture was chilled to 10° C. and stirred for 2 additional hours. 60.0 g (15% of the cyclodextrin weight) of gum acacia was added. Stirring continued for an additional hour. The mixture was then vacuum dried at 79° C. for 12 hours in a Heraeus Instruments vacutherm unit. The vacuum read approximately 1 mbar.

A percent retention of 3 wt % of blueberry flavor in the cyclodextrin inclusion complex was achieved. The moisture content was measured at 4%. The cyclodextrin inclusion complex included less than 0.05% surface blueberry flavor, and the particle size of the cyclodextrin inclusion complex was measured as 95% through a 10 mesh screen or 1500 microns, with greater than 50% holding on a 20 mesh screen (840 microns). Thus, the particle size was considered to be between 10 mesh (1500 microns) and 20 mesh (approximately 850 microns). Those skilled in the art will understand that heating and cooling can be controlled by other means.

### Example 4

### Formation of Large Particle Cyclodextrin Inclusion Complexes with Lemon Oil and Hardening Agent

The paste method employed in the following examples dramatically reduces the amount of water that needs to be removed in the drying process. The combination of reduced water, hardening agent, log(P) and drying conditions act synergistically to produce composite complexes of unique properties.

In an industrial mixer (Kitchen Aid Proline, Kitchen Aid, St. Joseph, Mich.), 1000.0000 g of β-cyclodextrin was mixed at low speed for 20 minutes with 700.0000 g of distilled water to form a paste in a dough mixture. 120.0000 g of lemon oil (SAP#0015551, available from Citrus&Allied, New Jersey) was added slowly while mixing. After 20 minutes the mixture was scrapped down and mixed for an additional 15 minutes. Almost no lemon odor was detected at this point.

Three 500 g samples were removed from the original mixer and different hardening agents were added. To the first sample (Sample 4A), 50 g of sucrose was added and the mixture was stirred for 10 minutes. To the second sample (Sample 4B), 75 g of EmCap® (SAP# 06438, a modified food starch available from Cargill) was added and the mixture was stirred for 10 minutes. To the third sample (Sample 4C), 75 g of gum acacia (SAP# 12265, available from Colloid Naturel) was added and the mixture was stirred for 10 minutes.

Samples 4A, 4B and 4C were vacuum dried at 79° C. for 12 hours. After drying, the samples were weighed directly onto a stack of 18 and 20 mesh screens and ground through the 18 mesh screen. For Sample 4A, 107.15 g (53.65%) held on the 20 mesh screen and 85.97 g (43.04%) passed through the 20 mesh screen. For Sample 4B, 132.36 g (66.18%) held on the 20 mesh screen and 65.44 g (32.72%) passed through the mesh screen. For Sample 4C, 123.12 g (61.72%) held on the 20 mesh screen and 69.55 g (34.87%) passed through the 20 mesh screen.

### Example 5

### Formation of Large Particle Cyclodextrin Inclusion Complexes with Lemon Oil and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste in a dough mixture. 120.0000 g of lemon oil (Citrus&Allied, New Jersey) and 0.12 g (0.1%) methyl jasmonate (Aldrich Chemical, Milwaukee, Wis.) were added slowly while mixing for 15 minutes. After 20 minutes, the mixture was scrapped down and mixed for an additional 15 minutes. Almost no lemon odor was detected at this point.

Two 500 g samples were removed from the original mixer and different hardening agents were added. To the first sample (Sample 5A), 50 g of sucrose was added and the mixture was stirred for 10 minutes. To the second sample (Sample 5B), 75 g of gum acacia was added and the mixture was stirred for 10 minutes.

Samples 5A and 5B were vacuum dried at 79° C. until a thermometer inserted into the paste reached the oven temperature of 79° C. After drying, the samples were weighed directly onto a stack of 18 and 20 mesh screen and ground through the 18 mesh screen. For Sample 5A, 134.7 g (67.35%) held on the 20 mesh screen and 66.15 g (33.08%) passed through the 20 mesh screen. For Sample 5B, 88.29 g (44.15%) held on the 20 mesh screen and 109.87 g (54.94%) passed through the 20 mesh screen.

It was noted that the sucrose containing large particle cyclodextrin inclusion complexes dissolved faster than the gum acacia containing large particle cyclodextrin inclusion complexes.

### Example 6

### Formation of Large Particle Cyclodextrin Inclusion Complexes with Lemon Oil and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste in a dough mixture. 75.0000 g of lemon oil (Citrus&Allied, New Jersey) was added slowly while mixing for 15 minutes.

Two samples of approximately 500 g were removed from the original mixer and different hardening agents were added. To the first sample (Sample 6A —571.02 g), 57.1 g (10%) of sucrose was added and the mixture was stirred for five (5) minutes. To the second sample (Sample 6B —507.73 g), 25.4 g (5%) of sucrose was added and the mixture was stirred for five (5) minutes.

Samples 6A and 6B were vacuum dried at 79° C. until a thermometer inserted into the paste reached the oven temperature of 79° C. The pans came out of the oven as a granular mixture, not as a cake. After drying, 200 g of each sample was weighed directly onto a 20 mesh screen and ground through the 20 mesh screen. For Sample 6A, 100% of the sample passed through the 20 mesh screen. For Sample 6B, 100% of the sample passed through the 20 mesh screen.

### Example 7

### Formation of Large Particle Cyclodextrin Inclusion Complexes with Lemon Oil and Hardening Agents

In an industrial mixer, (Kitchen Aid Proline, Kitchen Aid, St. Joseph, Mich.), 750.0000 g of β-cyclodextrin and 250.0000 of α-cyclodextrin were mixed at low speed for 20 minutes with 700.0000 g of distilled water to form a paste in a dough mixture. 75.0000 g of lemon oil (Citrus&Allied, New Jersey) was added slowly while mixing for 15 minutes.

Two samples of approximately 500 g were removed from the original mixer and different hardening agents were added. To the first sample (Sample 7A—554.1 g), 55.4 g (10%) of sucrose was added and the mixture was stirred for five (5) minutes. To the second sample (Sample 7B-521.8 g), 26.1 g (5%) of sucrose was added and the mixture was stirred for five (5) minutes.

Samples 7A and 7B were vacuum dried at 79° C. until a thermometer inserted into the paste reached the oven temperature of 79° C. After drying, 200 g of each sample was weighed directly onto a stack of 18 and 20 mesh screens and ground through the 18 mesh screen. For Sample 7A, 134.08 g (67.04%) collected on the 20 mesh screen. For Sample 7B, 145.54 g (72.77%) collected on the 20 mesh screen.

### Example 8

### Formation of Large Particle Cyclodextrin Inclusion Complexes with Bergamot and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste. 120.0000 g of bergamot oil (FW60550-9, available from Cargill-Duckworth Flavours, Manchester, UK) was added slowly while mixing for 20 minutes.

Two samples were removed from the original mixer and different hardening agents were added. To the first sample (Sample 8A—750.0 g), 75 g (10%) of sucrose was added and the mixture was stirred for five (5) minutes. To the second sample (Sample 8B—1070 g), 160 g (15%) of sucrose was added and the mixture was stirred for five (5) minutes.

Samples 8A and 8B were vacuum dried at 79° C. for 12 hours. After drying, the samples were weighed directly onto a stack of 18 mesh, 20 mesh and 40 mesh screens and ground through the 18 mesh screen. For Sample 8A, 450.3 g was ground through the 18 mesh screen, 325.7 g (72.3%) collected on the 20 mesh screen, 66.2 g (14.7%) collected on the 40 mesh screen, and 58.78 g (13.1%) went through the 40 mesh screen. For Sample 8B, 450.29 g was ground through the 18 mesh screen, 327.95 g (72.8%) collected on the 20 mesh screen, 56.10 g (12.5%) collected on the 40 mesh screen, and 65.85 g (14.6%) went through the 40 mesh screen.

### Example 9

### Formation of Large Particle Cyclodextrin Inclusion Complexes with Lemon Oil and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste in a dough mixture. 120.0000 g of lemon oil (FD60549-9, available from Cargill-Duclcworth Flavours, Manchester, UK) was added slowly while mixing for 15 minutes.

Two samples were removed from the original mixer and different hardening agents were added. To the first sample (Sample 9A—879.50 g), 10% by weight sucrose was added and the mixture was stirred for five (5) minutes. To the second sample (Sample 9B —1100 g), 154.65 g (15%) of sucrose was added and the mixture was stirred for five (5) minutes.

Samples 9A and 9B were vacuum dried at 79° C. for 8 hours. After drying, the samples were weighed directly onto a stack of 18 mesh, 20 mesh and 40 mesh screens and ground through the 18 mesh screen. For Sample 9A, 401.4 g was ground through the 18 mesh screen, 286.5 g (71.38%) collected on the 20 mesh screen, 71.09 g (17.71%) collected on the 40 mesh screen, and 48.69 g (12.15%) went through the 40 mesh screen. For Sample 9B, 451.87 g was ground through the 18 mesh screen, 387.5 g (85.75%) collected on the 20 mesh screen, 48.27 g (10.68%) collected on the 40 mesh screen, and 16.1 g (3.56%) went through the 40 mesh screen.

### Example 10

### Formation of Large Particle Cyclodextrin Inclusion Complexes with Peach Flavor and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste. 50.0000 g of peach flavor (FV60548-9, available from Cargill-Duckworth Flavours, Manchester, UK) was added slowly while mixing for 15 minutes.

Two samples were removed from the original mixer and different hardening agents were added. To the first sample (Sample 10A—803.00 g), 80.3 g (10%) sucrose was added and the mixture was stirred for five (5) minutes. To the second sample (Sample 10B —947 g), 142 g (15%) of sucrose was added and the mixture was stirred for five (5) minutes.

Samples 10A and 10B were vacuum dried at 79° C. for 6 hours. After drying, the samples were weighed directly onto a stack of 18 mesh, 20 mesh and 40 mesh screens and ground through the 18 mesh screen. For Sample 10A, 468.15 g was ground through the 18 mesh screen, 10.98 g (2.35%) collected on the 20 mesh screen, 71.3 g (15.28%) collected on the 40 mesh screen, and 383.68 g (81.96%) went through the 40 mesh screen. For Sample 10B, 603.54 g was ground through the 18 mesh screen, 32.0 g (5.3%) collected on the 20 mesh screen, 142.22 g (23.56%) collected on the 40 mesh screen, and 428.37 g (70.98%) went through the 40 mesh screen.

### Example 11

### Formation of Large Particle Cyclodextrin Inclusion Complexes with Lemon Oil, Pectin and Hardening Agents

In an industrial mixer, (Kitchen Aid Proline, Kitchen Aid, St. Joseph, Mich.), 1000.0000 g of β-cyclodextrin and 20.00 g (2.0 wt %) XPQ EMP 4 beet pectin (available from Degussa-France) were mixed at low speed for 5 minutes. 700.0000 g of distilled water was added with stirring to form a paste. 100.0000 g of lemon oil (011-0013, available from Cargill Flavor Systems, Cincinnati, Ohio) was added slowly and mixing continued for 30 minutes

Two samples were removed from the original mixer and different hardening agents were added. To the first sample (Sample 11A—600.00 g), 60 g (10%) sucrose was added and the mixture was stirred for five (5) minutes. To the second sample (Sample 11B—600 g), 90 g (15%) of sucrose was added and the mixture was stirred for five (5) minutes.

Samples 11A and 11B were vacuum dried at 79° C. for 8 hours. After drying, the samples were weighed directly onto a stack of 18 mesh, 20 mesh and 40 mesh screens and ground through the 18 mesh screen. For Sample 11A, 300.0 g was ground through the 18 mesh screen, 57.35 g (19.12%) collected on the 20 mesh screen, 145.8 g (48.6%) collected on the 40 mesh screen, and 95.4 g (31.8%) went through the 40 mesh screen. For Sample 11B, 300 g was ground through the 18 mesh screen, 73.18 g (24.66%) collected on the 20 mesh screen, 132.4 g (44.13%) collected on the 40 mesh screen, and 92.4 g (30.8%) went through the 40 mesh screen.

As can be seen in the above experiments, the particle size distribution can be dramatically impacted by log(P), the amount of guest flavor (which really is a log(P) contribution), pectin and the agents used in the hardening process.

### Example 12

### Formation of Large Particle Cyclodextrin Inclusion Complexes with Peppermint and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste in a dough mixture. 98.0000 g of peppermint flavor 086-03530 (available from Cargill Flavor Systems; Cincinnati, Ohio) was added slowly and mixing continued for 30 minutes.

Two samples were removed from the original mixer and different hardening agents were added. To the first sample (Sample 12A—800 g), 120 g (15%) sucrose was added and the mixture was stirred for five (5) minutes. To the second sample (Sample 12B—800 g), 120 g (15%) sorbitol was added and the mixture was stirred for five (5) minutes.

Samples 12A and 12B were vacuum dried at 79° C. for 8 hours. After drying, the samples were weighed directly onto a stack of 18 mesh, 20 mesh and 40 mesh screens and ground through the 18 mesh screen. For Sample 12A, 500.69 g was ground through the 18 mesh screen, 371.2 g (74.1%) collected on the 20 mesh screen, 81.17 g (16.2%) collected on the 40 mesh screen, and 46.4 g (9.27%) went through the 40 mesh screen. For Sample 12B, 500.19 g was ground through the 18 mesh screen, 365.02 g (72.98%) collected on the 20 mesh screen, 96.81 g (19.36%) collected on the 40 mesh screen, and 37.07 g (7.41%) went through the 40 mesh screen.

### Example 13

### Formation of Large Particle Cyclodextrin Inclusion Complexes with Spearmint and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste in a dough mixture. 60.0000 g of spearmint flavor 080-00706 (available from Cargill Flavor Systems; Cincinnati, Ohio) was added slowly and mixing continued for 30 minutes.

Two samples were removed from the original mixer and different hardening agents were added. To the first sample (Sample 13A—880 g), 132 g (15%) sucrose was added and the mixture was stirred for five (5) minutes. To the second sample (Sample 13B —746 g), 112 g (15%) sorbitol was added and the mixture was stirred for five (5) minutes.

Samples 13A and 13B were vacuum dried at 79° C. for 8 hours. After drying, the samples were weighed directly onto a stack of 18 mesh, 20 mesh and 40 mesh screens and ground through the 18 mesh screen. For Sample 13A, 500.1 g was ground through the 18 mesh screen, 25.54 g (5.1%) collected on the 20 mesh screen, 141.75 g (28.34%) collected on the 40 mesh screen, and 327.4 g (65.55%) went through the 40 mesh screen. For Sample 13B, 400.0 g was ground through the 18 mesh screen, minimal material collected on the 20 mesh screen and was ground through the 20 mesh screen, 138.61 g (34.65%) collected on the 40 mesh screen, and 231.23 g (65.3%) went through the 40 mesh screen.

### Example 14

### Formation of Large Particle Cyclodextrin Inclusion Complexes with Cocoa and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste in a dough mixture. 102.0000 g of Cocoa Absolute (available from Robertet; Oakland, N.J.) was added slowly while mixing for 30 minutes.

To 900.00 g of the above mixture 135.0 g or (15%) sucrose was added and the mixture was stirred for five (5) minutes. The sample was vacuum dried at 79° C. for 6.0 hours. After drying, the sample was ground through a 14 mesh screen to obtain a particle size similar to that of ground coffee. This product easily disperses in water and coffee bags and gives a strong cocoa impact to coffee. Most importantly, it disperses easily in the coffee beverage without plugging the coffee filters which had been a major issue when trying to employ cocoa powder, cocoa nibs, cocoa-chocolate liquors or pieces.

### Example 15

### Formation of Large Particle Cyclodextrin Inclusion Complexes with Cocoa and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste. 200.0000 g of Cocoa Absolute (available from Robertet; Oakland, N.J.) was added slowly while mixing for 30 minutes.

285 g (15%) sucrose was added and the mixture was stirred for five (5) minutes. The sample was vacuum dried at 79° C. for 6-8 hours. The sample was then removed from the oven and desiccated for 2 hours.

After drying, the sample was weighed directly onto a stack of 14 mesh, 18 mesh, 20 mesh and 40 mesh screens. 603.2 g was ground through the 14 mesh screen, 278.32 g (46.14%) collected on the 18 mesh screen, very little material collected on the 20 mesh screen and was ground through, 143.4 g (23.77%) collected on the 40 mesh screen, and 175.3 g (29.06%) was finer than 40 mesh. Only the larger particle (14-18 mesh) was used for further coffee applications.

### Example 16

### Formation of Large Particle Cyclodextrin Inclusion Complexes with Mint and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste. 100.0000 g of spearmint flavor 080-00706 (Cargill Flavor Systems, Cincinnati, Ohio) was added slowly and mixing continued for 30 to 60 minutes.

Two samples were removed from the original mixer and different hardening agents were added. To the first sample (Sample 16A of 900 g), 135 g (15%) sucrose was added and the mixture was stirred for five (5) minutes. To the second sample (Sample 16B of 900 g), 135 g (15%) sorbitol was added and the mixture was stirred for five (5) minutes.

Samples 16A and 16B were vacuum dried at 79° C. for six (6) to eight (8) hours. After drying, the samples were ground through an 80 mesh screen. The samples dissolved instantaneously in a mouth rinse formulation but maintain particle integrity in toothpaste formulations.

### Example 17

### Formation of Large Particle Cyclodextrin Inclusion Complexes with Cinnamon Flavor and Hardening Agents

In an industrial mixer, (Kitchen Aid Proline, Kitchen Aid, St. Joseph, Mich.), 1000.0000 g of β-cyclodextrin was mixed at low speed for 20 minutes with 700.0000 g of distilled water to form a paste. 100.0000 g of cinnamic aldehyde (SAP# 15499, Citrus+Allied, Lalce Success, N.Y.) was added slowly and mixing continued for 30-60 minutes.

Two samples were removed from the original mixer and different hardening agents were added. To the first sample (Sample 17A —900 g), 135 g (15%) sucrose was added and the mixture was stirred for five (5) minutes. To the second sample (Sample 17B —900 g), 135 g (15%) sorbitol was added and the mixture was stirred for five (5) minutes.

Samples 17A and 17B were vacuum dried at 79° C. for six (6) to eight (8) hours. After drying, the samples were ground through an 80 mesh screen.

### Example 18

### Formation of Large Particle Cyclodextrin Inclusion Complexes with Stevia-Derived Sweeteners and Hardening Agents

In an industrial mixer, (Kitchen Aid Proline, Kitchen Aid, St. Joseph, Mich.), 100.0000 g of β-cyclodextrin was mixed at low speed for 5 minutes with 2.00 g beet pectin (2.00% pectin, XPQ EMP 4 beet pectin available from Degussa-France). 70.0000 g of distilled water was added followed by 2.5000 g of a stevia-derived sweetener (M201, Cargill Minneapolis, Minn.) and 1.0 ml furaneol (4-hydroxy-2,5-dimethyl-3(2H) furanone FEMA # 3174 as a 15% furaneol in ethanol cut; (available from Alfrebro, a division of Cargill, Monroe, Ohio) were added slowly and mixing continued for an additional 45 minutes.

25 g of erythritol was added and the mixture was vacuum dried as previously described. After drying the composite complex is ground through an 18 mesh screen.

### Example 19

### Formation of Large Particle Cyclodextrin Inclusion Complexes with Stevia-Derived Sweeteners and Hardening Agents

In an industrial mixer, (Kitchen Aid Proline, Kitchen Aid, St. Joseph, Mich.), 50.0000 g of β-cyclodextrin, 50.0000 g of γ-cyclodextrin and 2.00 g beet pectin (2.00% pectin, XPQ EMP 4 beet pectin available from Degussa-France) were mixed at low speed for 5 minutes. 70.0000 g of distilled water was added followed by 2.5000 g of a stevia-derived sweetener (M201, Cargill Minneapolis, Minn.) and 1.0 ml furaneol (4-hydroxy-2,5-dimethyl-3(2H) furanone FEMA # 3174 as a 15% furaneol in ethanol cut; (available from Alfrebro, division of Cargill, Monroe, Ohio) were added slowly and mixing continued for an additional 45 minutes. 25 g of erythritol was added and the mixture stirred an additional five (5) minutes.

The sample was vacuum dried at 79° C. for 6 hours, as previously described and the composite complex ground through an 18 mesh screen. Upon sensory evaluation, the blend of cyclodextrins was judged superior to β-cyclodextrin alone in delivering high intensity sweetness and masking bitter attributes in coffee, toothpaste and mouth rinse products.

### Example 20

### Formation of Large Particle Cyclodextrin Inclusion Complexes with Stevia-Derived Sweeteners and Hardening Agents

In an industrial mixer, (Kitchen Aid Proline, Kitchen Aid, St. Joseph, Mich.), 100.0000 g of β-cyclodextrin, 100.0000 g of γ-cyclodextrin and 4.00 g beet pectin (2.0% pectin, XPQ EMP 4 beet pectin available from Degussa-France) were mixed at low speed for 5 minutes. 120.0000 g of distilled water was added. 10.0 g of a stevia-derived sweetener (5%) (M201, Cargill Minneapolis, Minn.) and 1.0 ml furaneol (4-hydroxy-2,5-dimethyl-3(2H) furanone FEMA # 3174 as a 15% furaneol in ethanol cut (available from Alfrebro, division of Cargill, Monroe, Ohio) were added slowly and mixing continued for an additional 45 minutes. 50.00 g (25 wt %) erythritol was added and the mixture was stirred for an additional five (5) minutes.

The sample was vacuum dried at 79° C. for 6 hours. After drying, the sample was weighed directly onto a stack of 18 mesh, 20 mesh and 40 mesh screens. 94 g was ground through the 18 mesh screen, very little material collected on the 20 mesh screen and was ground through; 59.66 g (63.5%) collected on the 40 mesh screen, and 33.6 g (35.7%) was finer than 40 mesh. The major portion (63.5%) of the composite complex has the desired sensory and visual properties for table top use.

### Example 21

### Formation of Large Particle Cyclodextrin Inclusion Complexes with Menthol

In an industrial mixer, (Kitchen Aid Proline, Kitchen Aid, St. Joseph, Mich.), 100.0000 g of β-cyclodextrin, 100.0000 g of γ-cyclodextrin and 4.0 g beet pectin (2.0% pectin, XPQ EMP 4 beet pectin available from Degussa-France) were mixed at low speed for 5 minutes. 120.0000 g of distilled water was added. 10.0000 g of menthol (FEMA# 2665 available from Penta, Livingston, N.J.) was dissolved in 10.0 g ethanol. The menthol-ethanol solution was added slowly while mixing for 30-40 minutes.

The sample was vacuum dried at 79° C. for 6 hours. After drying, the sample was ground through an 80 mesh screen.

### Example 22

### Formation of Large Particle Cyclodextrin Inclusion Complexes with Menthol

In an industrial mixer, (Kitchen Aid Proline, Kitchen Aid, St. Joseph, Mich.), 100.0000 g of β-cyclodextrin, 100.0000 g of γ-cyclodextrin and 4.00 g beet pectin (2.00% pectin, XPQ EMP 4 beet pectin available from Degussa-France) were mixed at low speed for 5 minutes. 120.0000 g of distilled water was added. 10.0000 g of menthol (FEMA # 2665 available from Penta, Livingston, N.J.) was dissolved in 10.0 g ethanol. The menthol-ethanol solution was added slowly while mixing. Additionally, 5.00 g Glyceryzinate (a sapponin) (FEMA # 2528; available from MAFCO Camden, N.J.) was added; mixing was continued for 30-40 minutes.

The sample was vacuum dried at 79° C. for 6 hours. After drying, the sample was ground through and 80 mesh screen. This preparation is useful in mouth rinse formulations.

### Example 23

### Formation of Large Particle Cyclodextrin Inclusion Complexes with Stevia-Derived Sweeteners and Hardening Agents

In an industrial mixer, (Kitchen Aid Proline, Kitchen Aid, St. Joseph, Mich.), 100.0000 g of β-cyclodextrin and 100.0000 g of γ-cyclodextrin were mixed at low speed for 5 minutes. 120.0000 g of distilled water was added. 10.0 g of a stevia-derived sweetener (5%)(M201, Cargill Minneapolis, Minn.) and 2.0 ml furaneol (4-hydroxy-2,5-dimethyl-3(2H) furanone FEMA # 3174 as a 15% furaneol in ethanol cut (available from Alfrebro, division of Cargill, Monroe, Ohio) were added slowly and mixing continued for an additional 45 minutes. 50.0 g (25%) erythritol was added and the mixture was stirred for an additional five (5) minutes.

The sample was vacuum dried at 79° C. for 6 hours. The vacuum was vented slightly several times during drying to control foaming. After drying, the sample was weighed directly onto a stack of 20 mesh, 40 mesh and 80 mesh screens. 200 g was ground through the 20 mesh screen, 101.02 g (50.6%) collected on the 40 mesh screen, 50.03 g (25.02%) collected on the 80 mesh screen, and 48.43 g (24.22%) was finer than 80 mesh.

### Example 24

### Formation of Large Particle Cyclodextrin Inclusion Complexes with Cinnamic Aldehyde

In an industrial mixer, (Kitchen Aid Proline, Kitchen Aid, St, Joseph, Mich.), 100.0000 g of β-cyclodextrin, 100.0000 g of γ-cyclodextrin and 4.0 g beet pectin (2.0% pectin, XPQ EMP 4 beet pectin available from Degussa-France) were mixed at low speed for 5 minutes. 120.0000 g of distilled water was added. 11.0 g cinnamic aldehyde (FEMA # 2286; available from, Citrus+Allied, Lake Success, N.Y.) was added slowly while mixing for 30-40 minutes. The sample was vacuum dried at 79° C. for 6 hours and ground to an 80 mesh composite complex and used as a flavor key or ingredient in tooth paste, mouth rinse, chewing gums and candies.

### Example 25

### Formation of Large Particle Cyclodextrin Inclusion Complexes with Lemon Oil

In an industrial mixer, (Kitchen Aid Proline, Kitchen Aid, St. Joseph, Mich.), 400.0000 g of β-cyclodextrin, 0.65 g or 0.05% of the total mixture Keltrol brand xanthan gum (CP Kelco, Chicago, Ill.) and 8.00 g beet pectin (XPQ EMP 4 beet pectin available from Degussa-France) were mixed at low speed for five (5) minutes. 300.0000 g of distilled water was added. 25.0 g citrus topnote VML 00401-001 (an experimental flavor formulation) was added slowly and mixing continued for 60 minutes. An additional 500.0000 g of distilled water was added and the material was stirred for five (5) minutes. The resulting mixture is 33.33% solids. The sample was spray dried.

### Example 26

### Formation of Large Particle Cyclodextrin Inclusion Complexes with Cinnamon and Hardening Agent

In an industrial mixer, (Kitchen Aid Proline, Kitchen Aid, St. Joseph, Mich.), 200.0000 g of β-cyclodextrin, and 4.0 g beet pectin (2.0% pectin, XPQ EMP 4 beet pectin available from Degussa-France) were mixed at low speed for 5 minutes. 120.0000 g of distilled water was added. 29.4 g cinnamon flavor 125-01934 and 0.63 g cinnamon flavor 125-01935 (both available from Cargill Flavors, Cincinnati, Ohio) were added slowly and mixing continued for 30-40 minutes. As the final step, 35 g sorbitol was added with mixing for five (5) minutes. The sample was vacuum dried at 78° C. for 8 hours. The sample was ground through a 40 mesh screen. Yield 224.53 g. (96.2%)

### Example 27

### Formation of Large Particle Cyclodextrin Inclusion Complexes with Cinnamon and Hardening Agent

In an industrial mixer, (Kitchen Aid Proline, Kitchen Aid, St. Joseph, Mich.), 200.0000 g of β-cyclodextrin, and 4.0 g beet pectin (2.0% pectin, XPQ EMP 4 beet pectin available from Degussa-France) were mixed at low speed for 5 minutes. 120.0000 g of distilled water was added. 30.0 g cinnamon flavor USL-44163 (available from Cargill Flavors, Cincinnati, Ohio) was added slowly while mixing for 30-40 minutes. 35 g (15%) sorbitol was added to complete the formulation. The sample was vacuum dried at 78° C. for 8 hours. The sample was ground through a 40 mesh screen. Yield 204.45 g. (87.4%). This formulation is used in tooth paste applications.

### Example 28

### Formation of Large Particle Cyclodextrin Inclusion Complexes with Apple Flavor and Hardening Agent

In an industrial mixer, (Kitchen Aid Proline, Kitchen Aid, St. Joseph, Mich.), 200.0000 g of β-cyclodextrin, and 4.0 g beet pectin (2.0% pectin, XPQ EMP 4 beet pectin available from Degussa-France) were mixed at low speed for 5 minutes. 140.0000 g of distilled water was added. 30.0000 g apple flavor (Granny Smith type) (060-02253 available from Cargill Flavor Systems, Cincinnati, Ohio) was added slowly while mixing for 30-40 minutes. 35 g (15%) sorbitol was added. The sample was vacuum dried at 78° C. for 8 hours. The sample was ground through a 40 mesh screen. Yield 199.56 g (85.3%). This formulation is being evaluated in tooth paste.

### Example 29

### Formation of Large Particle Cyclodextrin Inclusion Complexes with Apple Flavor and Hardening Agent

In an industrial mixer, (Kitchen Aid Proline, Kitchen Aid, St. Joseph, Mich.), 200.0000 g of β-cyclodextrin, and 4.0 g beet pectin (2.0% pectin, XPQ EMP 4 beet pectin available from Degussa-France) were mixed at low speed for 5 minutes. 140.0000 g of distilled water was added. 30.0000 g apple flavor (060-04159, available from Cargill Flavor Systems, Cincinnati, Ohio) was added slowly, with mixing continued for 30-40 minutes. 35 g (15%) sorbitol was added. The sample was vacuum dried at 78° C. for 8 hours. The sample was ground through a 40 mesh screen. Yield 194.15 g. (82.97%). This formulation is being evaluated in tooth paste.

### Example 30

### Formation of Large Particle Cyclodextrin Inclusion Complexes with Lemon Flavor and Hardening Agent

In an industrial mixer, (Kitchen Aid Proline, Kitchen Aid, St. Joseph, Mich.), 750.0000 g of β-cyclodextrin, and 15.00 g beet pectin (2.00% pectin, XPQ EMP 4 beet pectin available from Degussa-France) were mixed at low speed for five (5) minutes. 500.0000 g of distilled water was added and the mixture was stirred for 2 minutes. 100.0000 g lemon flavor 125-01984 (available from Cargill Flavor Systems, Cincinnati, Ohio) was added slowly while mixing for 15 minutes. As with all previous examples, the odor of the guest molecule or flavor will disappear, as complexation is complete.

Two samples were removed from the original mixer and different hardening agents were added. To the first sample (Sample 30A—500 g), 75 g or 15% sucrose was added and the mixture was stirred for five (5) minutes. To the second sample (Sample 30B—500 g), 75 g or 15% citric acid was added and the mixture was stirred for five (5) minutes. The samples were vacuum dried as previously described at 78° C. for 8 hours. 400.8 g of Sample 30A and 300.04 g of Sample 30B were ground through a 40 mesh screen; the yield of Sample 30A was 250.21 g (62.4%) and Sample 30B was 176.79 g (58.92%).

### Example 31

### Formation of Large Particle Cyclodextrin Inclusion Complexes with Neohesperidin Dihydrochalcone

In an industrial mixer, (Kitchen Aid Proline, Kitchen Aid, St. Joseph, Mich.), 200.0000 g of β-cyclodextrin, 140.0000 g of distilled water was added. 25.0 g neohesperidin dihydrochalcone FEMA# 3811 (Penta: Livingston, N.J.) was added slowly while mixing for 30-40 minutes. The sample was vacuum dried at 79° C. for 6 hours. The sample was ground through an 80 mesh screen.

### Example 32

### Use in Mouth Rinse

A cyclodextrin-encapsulated spearmint flavor produced according to Example 13 was incorporated into a mouth rinse at a 0.2% by weight of the product and at a 10:1 dilution in additional β-cyclodextrin at 0.05% to 0.1% by weight of the product.

### Example 33

### Use in Toothpaste

A cyclodextrin-encapsulated spearmint flavor produced according to Example 13 was incorporated into CREST PRO HEALTH toothpaste (Proctor & Gamble, Cincinnati Ohio) at 0.1% by weight of the product. The resulting product had a boosted freshness and an extended mint profile. In addition, the product had a reduced medicinal offnote.

### Example 34

### Use in TEA

A cyclodextrin-encapsulated lemon flavor produced according to Example 30 was incorporated into brewed LIPTON tea (Unilever) at 0.06% by weight of the product. The resulting product had a true fresh squeezed lemon character. The citric acid containing lemon flavor had a truer fresh squeezed lemon character than the sucrose containing lemon flavor.

### Example 35

### Use in Coffee

A cyclodextrin-encapsulated cocoa flavor produced according to Example 15 was incorporated into an instant coffee product at 0.2% by weight of the product. The resulting product had a great aroma and a dark semi-sweet chocolate profile lingering through the aftertaste.

### Example 36

### Use in Mouth Rinse

A cyclodextrin-encapsulated spearmint flavor produced according to Example 13 was combined with a sweetener from Example 31 and incorporated into a mouth rinse product at 0.1% mint flavor by weight of the product and 0.1% sweetener by weight of the product.

### Example 37

### Use in Mouth Rinse

A cyclodextrin-encapsulated spearmint flavor produced according to Example 13 is combined with a sweetener from Example 31 and incorporated into a CREST PRO HEALTH mouth rinse product (Proctor & Gamble, Cincinnati, Ohio) at 0.1% mint flavor by weight of the product and 0.1% sweetener by weight of the product.

### Example 38

### Use in Coffee

A cyclodextrin-encapsulated cocoa flavor produced according to Example 15 is incorporated into GENERAL MILLS INTERNATIONAL coffee (Kraft Foods, Illinois) at 0.2% by weight of the product.

### Example 39

### Use in Tea

A cyclodextrin-encapsulated lemon flavor produced according to Example 30 is incorporated into LIPTON tea (Unilever) at 0.06% by weight of the product.

All patents, publications and references cited herein are hereby fully incorporated by reference. In case of conflict between the present disclosure and incorporated patents, publications and references, the present disclosure should control.

What is claimed is:

1. A method of imparting flavor to a product to form a flavored product, the method comprising: incorporating a large particle cyclodextrin inclusion complex into a product to form a flavored product, the complex comprising a guest encapsulated by a cyclodextrin.

2. The method of claim 1, wherein the large particle cyclodextrin complex is greater than about 500 microns in size.

3. The method of claim 1, wherein the large particle cyclodextrin complex is greater than about 800 microns in size.

4. The method of claim 1, wherein the guest includes at least one of a flavor, an olfactant, a pharmaceutical agent, a nutraceutical agent, and a combination thereof.

5. The method of claim 4, wherein the flavor includes at least one of an aldehyde, a ketone, an alcohol, and a combination thereof.

6. The method of claim 4, wherein the olfactant includes at least one of natural fragrances, synthetic fragrances, synthetic essential oils, natural essential oils, and a combination thereof.

7. The method of claim 1, wherein the guest includes at least one of fatty acids, lactones, terpenes, diacetyl, dimethyl sulfide, proline, furaneol, linalool, acetyl propionyl, natural essences, essential oils, and a combination thereof.

8. The method of claim 1, wherein the guest includes diacetyl.

9. The method of claim 1, wherein the flavored product includes at least one dentifrices, beverages, french fries, breadings, batter, pizza crust, pizza dough, and pizza sauce.

10. The method of claim 9, wherein the flavored product comprises a dentifrice.

11. The method of claim 10, wherein the dentifrice comprises toothpaste.

12. The method of claim 10, wherein the dentifrice comprises a mouth rinse.

13. The method of claim 10, wherein the guest includes at least one of mint flavors, cinnamon flavors and apple flavors.

14. The method of claim 13, wherein the mint flavor includes at least one of peppermint and spearmint.

15. The method of claim 9, wherein the flavored product comprises a beverage.

16. The method of claim 15, wherein the beverage comprises tea.

17. The method of claim 16, wherein the guest includes at least one of lemon flavors and bergamot flavors.

18. The method of claim 15, wherein the beverage comprises coffee.

19. The method of claim 18, wherein the guest comprises a cocoa flavor.

20. The method of claim 1, wherein the cyclodextrin comprises α-cyclodextrin.

21. The method of claim 1, wherein the cyclodextrin comprises β-cyclodextrin.

22. The method of claim 1, wherein the cyclodextrin comprises γ-cyclodextrin.

23. The method of claim 1, wherein the flavored product has a non-linear flavor delivery.

24. The method of claim 1, wherein the flavored product has a sequential flavor delivery.

25. The method of claim 1, wherein the flavored product has visible flavor particles.

26. The method of claim 1, wherein the flavored product contains about 0.001% to about 5% by weight of the cyclodextrin inclusion complex.

27. A cyclodextrin inclusion complex comprising a guest encapsulated by cyclodextrin, the complex being greater than about 400 microns in size.

28. The cyclodextrin inclusion complex of claim 27, wherein the ratio of guest to cyclodextrin is about 0.2:1 to about 2:1.

29. The cyclodextrin inclusion complex of claim 27, wherein the ratio of guest to cyclodextrin is about 1:1.

30. A flavored product comprising the cyclodextrin inclusion complex of claim 27.

31. A dentifrice comprising the cyclodextrin inclusion complex of claim 27.

32. The dentifrice of claim 31, wherein the cyclodextrin inclusion complex comprises a guest selected from the group consisting of mint flavors, cinnamon flavors and apple flavors.

33. A toothpaste comprising the cyclodextrin inclusion complex of claim 27.

34. A mouth rinse comprising the cyclodextrin inclusion complex of claim 27.

35. A tea product comprising the cyclodextrin inclusion complex of claim 27.

36. The tea product of claim 35, wherein the cyclodextrin inclusion complex comprises a guest selected from the group consisting of lemon flavors and bergamot flavors.

37. A coffee product comprising the cyclodextrin inclusion complex of claim 27.

38. The coffee product of claim 37, wherein the cyclodextrin inclusion complex comprises a guest comprising a cocoa flavor.

39. A sweetener comprising the cyclodextrin complex of claim 27.

40. A method of making a large particle cyclodextrin inclusion complex comprising: (a) mixing cyclodextrin with solvent to form a first mixture; (b) adding a guest to the first mixture to form a second mixture; (c) adding a hardening agent to the second mixture to form a third mixture; and (d) drying the third mixture to form a large particle cyclodextrin inclusion complex.

41. The method of claim 40, wherein the cyclodextrin to solvent ratio is from about 30:70 to about 70:30.

42. The method of claim 40, wherein the cyclodextrin to solvent ratio is from about 45:55 to about 65:35.

43. The method of claim 40, wherein the cyclodextrin to solvent ratio is from about 50:50 to about 60:40.

44. The method of claim 40, wherein the solvent comprises water.

45. The method of claim 40, wherein the hardening agent comprises sucrose.

46. The method of claim 40, wherein the hardening agent comprises gum acacia.

47. The method of claim 40, wherein the hardening agent comprises starch.

48. The method of claim 40, wherein the hardening agent comprises sorbitol.

49. The method of claim 40, wherein the hardening agent is present in an amount from about 5% to about 35% by weight of the cyclodextrin.

50. The method of claim 40, further comprising mixing an emulsifier with the cyclodextrin prior to forming the first mixture.

51. The method of claim 50, wherein the emulsifier comprises at least one of xanthan gum, pectin, gum acacia, tragacanth, guar, carrageenan, locust bean, and a combination thereof.

52. The method of claim 50, wherein the emulsifier comprises pectin.

53. The method of claim 52, wherein the pectin includes at least one of beet pectin fruit pectin, and a combination thereof.

54. The method of claim 40, further comprising milling the dry cyclodextrin inclusion complex.

55. The method of claim 40, wherein the large particle cyclodextrin complex is greater than about 500 microns in size.

56. The method of claim 40, wherein the large particle cyclodextrin complex is greater than about 800 microns in size.

57. The method of claim 40, wherein drying includes at least one of air drying, vacuum drying, spray drying, oven drying, and a combination thereof.
